(19) United States
(12) Patent Application Publication
(10) Pub. No.: US 2023/0149522 A1
(43) Pub. Date: May 18, 2023

(54) POLYPEPTIDE FOR THE PROPHYLAXIS AND TREATMENT OF VIRAL INFECTIONS

(71) Applicant: PHARIS BIOTEC GMBH, Hannover (DE)

(72) Inventors: Wolf-Georg Forssmann, Hannover (DE); Hans-Jürgen Mägert, Köthen (DE)

(21) Appl. No.: 17/915,812

(22) PCT Filed: Mar. 29, 2021
(86) PCT No.: PCT/EP2021/058150

§ 371 Date: Sep. 29, 2022

(30) Foreign Application Priority Data
Mar. 31, 2020 (WO) 20167370.4
Jul. 7, 2020 (WO) 20184401.6

(51) Int. Cl. A61K 38/57; A61P 31/14

(52) U.S. Cl. CPC A61K 38/57; A61P 31/14

(57) ABSTRACT

Polypeptide having an amino acid sequence selected from at least 70% sequence identity to any one of domains 1 to 14 of LEKTI, SPINK6 or SPINK9-polypeptide for use in the treatment of a viral infection with a virus, in the infection of which serine proteinases are involved, and slowing down the spread of infection of said virus.

The subject matter of the present invention is a polypeptide for the prophylaxis and treatment of viral infections with a virus whose infection involves serine proteinases, in particular coronaviruses, such as CoV-2 or influenza viruses.

SARS-CoV-2 is the name of a new coronavirus identified in January 2020 in the Chinese city of Wuhan, Hubei province. The virus causes the disease called COVID-19 and is the cause of the worldwide COVID-19 pandemic. Coronavirus disease (COVID-19) is an infectious disease caused by the novel virus SARS-CoV-2, identified for the first time in humans. The virus causes, among other things, a respiratory illness with the symptoms of a cough and fever. Pneumonia may occur in more severe cases. The pandemic caused by the coronavirus has led to extremely serious health problems worldwide and, consequently, to considerable economic and social disruption.

Influenza viruses belong to the Orthomyxoviridae family (RNA viruses) and cause the disease influenza (“real” flu).

WO 02/066513 A2 and WO 03/070953 A1 reveal human circulating fragments of the serine proteinase inhibitor LEKTI and their use for the treatment of acute or chronic skin diseases, cervical inflammations, inflammations of the Bartholinian glands and other vaginal areas, tonsillitis, pharyngitis and laryngitis, acute or chronic inflammatory processes associated with excessive mucus formation and resulting acute emergency situations, postoperative bleeding due to hyperfibrinolysis, for the prophylaxis of pulmonary emphysema formation in α1-proteinase inhibitor deficiency, and for the therapy of asthma, AIDS, pneumonia, tumour disease and leukaemia. LEKTI is the acronym for Lympho-epithelial Kazal-type-related inhibitor (LEKTI) and also known as serine protease inhibitor Kazal-type 5 (SPINK5) and is encoded in humans by the gene SPINK5. The polypeptides SPINK6 and SPINK9 are also known.

There is an urgent need to provide the means to slow down or stop the spread of the virus as a prophylactic measure on the one hand and to enable therapeutic treatment of infected patients on the other.

Surprisingly, it was found that the LEKTI domains 1 to 14, in particular 3 to 14, and also the polypeptides SPINK6 and SPINK9 act as peptidic inhibitors of TMPRSS2 and KLK5 and can be used prophylactically and therapeutically for the treatment of viral infections or diseases caused thereby, such as COVID-19 or influenza. Polypeptides have at least a 70% sequence identity.

TMPRSS2 (epitheliasin) is an endogenous trypsin-like serine proteinase (protein-hydrolysing enzyme) essential for infection by coronavirus-2 or influenza viruses. Without being tied to a theory, the mechanistic principle might be imagined as follows: TMPRSS2 cleaves a part of a “spike” protein of the virus hydrolytically, which activates it and allows it to dock to certain surface proteins of the host cells. This is an essential prerequisite for infection. An inhibitor of this proteinase therefore represents a drug for the treatment of COVID-19 or influenza (Li Wen Shen, Hui Juan Mao, Yan Ling Wu, Yoshimasa Tanaka, Wen Zhang, TMPRSS2: A potential target for treatment of influenza virus and coronavirus infections, Biochimie 142 (2017) 1-10; Brian S. Hamilton and Gary R. Whittaker, Cleavage Activation of Human-adapted Influenza Virus Subtypes by Kallikrein-related Peptidases 5 and 12*, The Journal Of Biological Chemistry Vol. 288, No. 24, pp. 17399-17407, Jun. 14, 2013). The trypsin-like serine protease KLK5 (kallikrein-related peptidase 5) can cleave after a lysine residue, preferably cleaving after an arginine (MEROPS database http://www.ebi.ac.uk/merops/cgi-bin/pepsum?id=S01.017;type=P). Due to the comparable properties of KLK5 and TMPRSS2 in the activation of influenza spike proteins, it is assumed that both have a comparable cleavage specificity and that TMPRSS2 also prefers to cleave after an arginine.

LEKTI domains 3 to 14 in the inhibitory centre, which “imitates” the natural substrates, have an arginine residue at the corresponding site (“P1 site”). Thus, LEKTI domains 3 to 14 in particular are promising inhibitors of the serine proteinases KLK5 and TMPRSS2 and hence of influenza and coronavirus infection.

The subject matter of the invention is consequently, first, a polypeptide having an amino acid sequence selected from at least one of the domains 1 to 14 of the LEKTI polypeptide, in particular a polypeptide having an amino acid sequence of SEQ ID Nos 1 to 35 and, second, the polypeptides SPINK6 or SPINK9, in particular having the SEQ ID No 37 and 39 or 40 and 42, for use in the treatment of a viral infection with a virus, in the infection of which serine proteinases are involved, and slowing down the spread of infection of said virus, in particular a coronavirus or influenza virus.

In a further embodiment, the present invention refers to the use of a polypeptide having an amino acid sequence selected from at least 70% sequence identity to any one of domains 1 to 14 of LEKTI, SPINK6 or SPINK9-polypeptide for manufacturing a medicament in the treatment of a viral infection with a virus, in the infection of which serine proteinases are involved, and slowing down the spread of infection of said virus.

In still a further embodiment, the present invention refers to a method for treatment of a disease caused by a viral infection with a virus, in the infection of which serine proteinases are involved, and slowing down the spread of infection of said virus, by administering a polypeptide having an amino acid sequence selected from at least 70% sequence identity to any one of domains 1 to 14 of LEKTI, SPINK6 or SPINK9-polypeptide.

These embodiments are further explained in the following. All preferred features can be applied in any combination to any of the above embodiments.

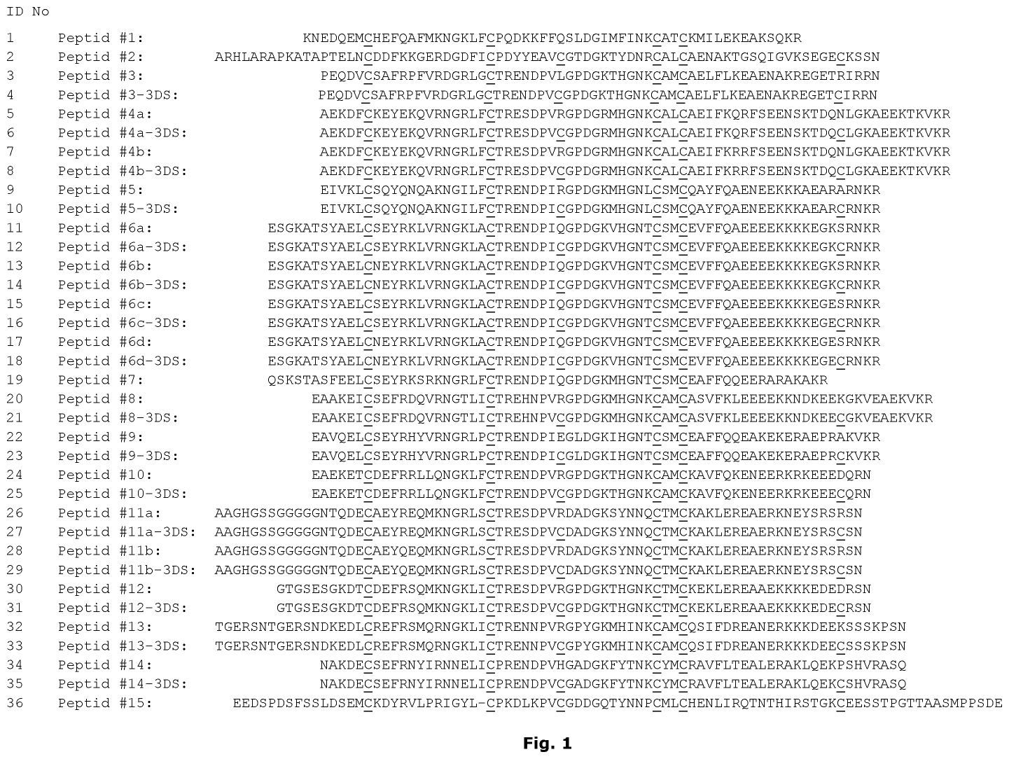

FIG. 1 shows an alignment of typical representatives of the 15 domains of the LEKTI polypeptide. Typical is the respective presence of four Cys residues in the amino acid sequence, whereas the presence of six cysteines shows an enhancement of the inhibition effect. The spacing of the Cys residues is the same in the other domains, with the exception of peptide 15, where a deletion is required in the alignment before the second Cys for a match, meaning that the identical number of amino acids is present there. Conspicuous in the region between the second and third Cys is the -TREN/SD- motif, which is conserved in most domains.

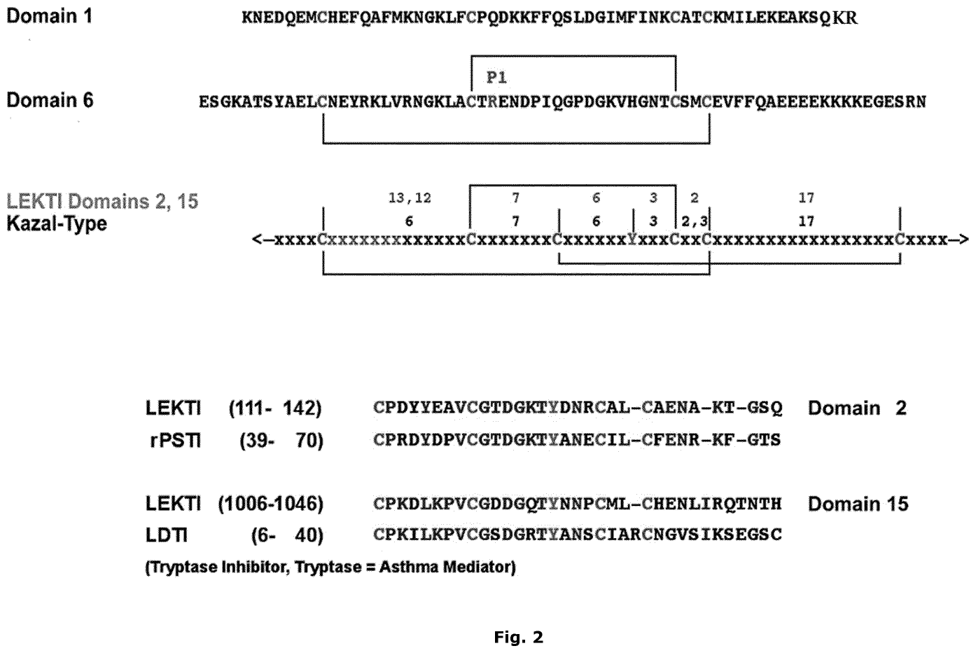

FIG. 2 shows the typical bridging of the cysteines in the domains when four and six cysteines respectively are present in the amino acid sequence. If four cysteines are present, the first cysteine is bridged with the fourth and the second cysteine with the third. If six cysteines are present, the formation of S—S bridges typically occurs between the first and fifth, between the second and fourth, and between the third and sixth cysteines.

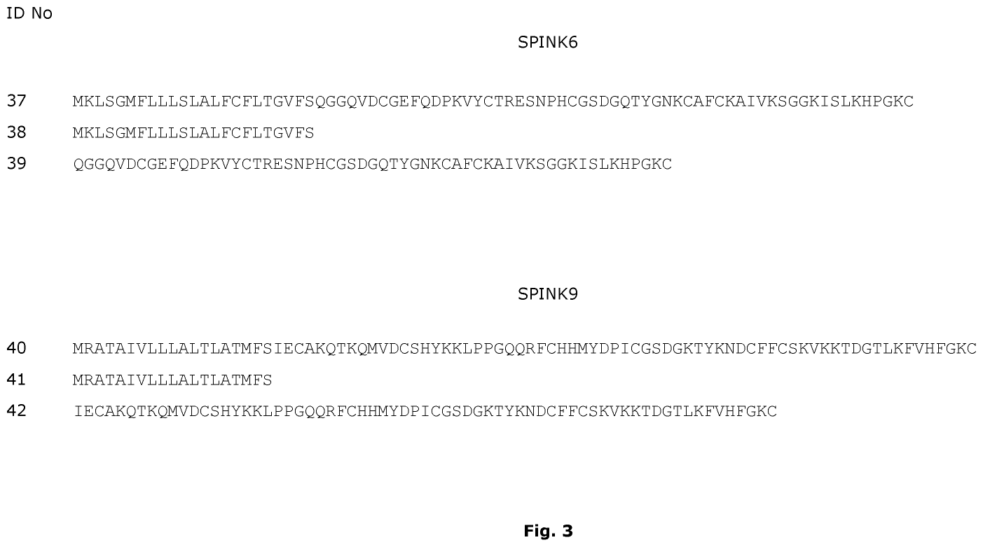

FIG. 3 shows the so-called P1 site for domains 1 to 15 of the Lekti.

FIG. 4 shows the sequences of the amino acids of the polypeptides SPINK6 (https://www.ncbi.nlm.nih.gov/protein/NP_995313.2) and SPINK9 (https://www.ncbi.nlm.nih.gov/protein/NP_001035523.1).

The mature SPINK6 protein (SEQ ID No 39) ranges from amino acid 24-80 of SEQ ID No 37. Amino acids 1-23 of SEQ ID No 38 correspond to the secretory signal peptide.

The mature SPINK9 protein (SEQ ID No 42) ranges from amino acid 20-86 of SEQ ID No 40. Amino acids 1-19 of SEQ ID No 41 correspond to the secretory signal peptide.

In one embodiment of the invention, the polypeptide is a homo- or heterodimer from the domains of the LEKTI, SPINK6 or SPINK9 polypeptide. For example, a polypeptide of LEKTI domain 2 may be linked to a polypeptide of LEKTI domain 3. In this case, a heterodimer of domains 2 and 3 is present. A homodimer is present if, for example, two domains 5 are connected to each other. The domains can be connected, for example, via a peptide bond between the N-terminus of one domain and the N-terminus of the other domain. Other intermolecular linkages are also possible.

In one embodiment of the invention, the polypeptide comprises LEKTI, SPINK6 or SPINK9 sequences of porcine, bovine origin, or LEKTI forms of primates, in particular humans.

In another embodiment of the invention, the polypeptide is a LEKTI, SPINK6 or SPINK9 fragment possessing therapeutic activity, a mutant and/or a derivative of the human LEKTI domains, SPINK6 or SPINK9.

LEKTI, SPINK6 or SPINK9 variants also include modified forms of the polypeptide as well as mutants or derivatives thereof. Modified LEKTI, SPINK6 or SPINK9 are polypeptides in which one or more amino acids of the native sequence have been modified so that a non-naturally occurring amino acid residue is present in the polypeptide chain. Such modifications can in principle be performed during or after protein translation and include, for example, phosphorylation, glycosylation, sulphonation, cross-linking, acylation or proteolytic cleavage.

In addition to the LEKTI, SPINK6 or SPINK9 polypeptide, derivatives, variants and fragments thereof are also useful as agents against corona infection, provided that they produce inhibition of infection by a virus in the infection of which serine proteinases are involved, such as a coronavirus or influenza virus, comparable to that of the native LEKTI, SPINK6 or SPINK9 fragment.

Amino acid substitutions can typically be made in a conservative manner. Conservative substitution refers to a mutation in which one codon is replaced by another. In this case, a different amino acid is encoded, which is, however, chemically related to the original amino acid, for example glycine to alanine or threonine to serine. A non-conservative substitution, on the other hand, occurs when the original codon is replaced by a codon encoding an amino acid with different chemical properties, for example glycine with lysine.

The terms “identical” or percent “identical” in the context of two or more polypeptides refer to two or more sequences or subsequences that are the same or have a certain percentage of amino acids that are identical when compared and “aligned” for maximum similarity using a sequence comparison algorithm. Optimised alignment for comparing sequences can be performed using, for example, the following algorithms: Local Homology Alignment of Smith &Waterman, Adv. Appl. Math. 2: 482 (1981), Homology Alignment Algorithm by Needleman & Wunsch, J. Mol. Biol. 48: 443 (1970), Pearson & Lipman, Proc. Nat'l. Acad. Sci. USA 85: 2444 (1988), (GAP, BESTFIT, FASTA, and TFASTA in Wisconsin Genetics Software Package, Genetics Computer Group, 575 Science Dr., Madison, Wis.), but also visual examination [compare, Current Protocols in Molecular Biology, (Ausubel, F. M. et al., eds.) John Wiley & Sons, Inc, New York (1987-1999, including Supplement 46 (April 1999)].

The expression “substantially identical” or similar expressions used in the context of two polypeptides refers to two or more sequences or subsequences that have at least 70%, in particular at least 80%, in particular at least 90%, in particular at least 95% sequence identity when compared and aligned for maximum similarity using a sequence comparison algorithm. Substantial identity exists in particular when there is a sequence region which is at least 40-60 amino acids in length, especially 60-80 amino acids, preferably more than 90-100 amino acid residues, and in particular is substantially identical over the entire length of the amino acid sequence of the native polypeptide.

Fragments of the polypeptide can typically be obtained by cleaving the polypeptide chain of the corresponding domain of the LEKTI, SPINK6 or SPINK9 polypeptide. Methods for fragmentation by cleavage of the polypeptide chain are known to an expert.

For example, polypeptides can be cleaved by enzymes known as proteases. A distinction is made between proteinases (also called endopeptidases) depending on whether the proteases cleave polypeptides (proteins) within the amino acid chain or at the end. They cleave proteins within the amino acid chain. They mostly recognise specific sequence segments within a protein where they can attack and cleave there.

On the other hand, exoproteases (also called exopeptidases) cleave individual amino acids from the ends of the protein. In contrast to proteinases, they mainly break down smaller peptides and not proteins. A distinction is made between:
Carboxypeptidases, which cleave amino acids from the carboxyl end (C-terminus) and
Aminopeptidases that cleave amino acids from the amino end (N-terminus).

Specific proteases cleave the peptide bond between two specific amino acids; in some cases, they recognise more than one substrate. These proteases are used, for example, to investigate the affinity of proteins, to prepare proteins for sequencing and to isolate active domains of a protein. Some examples of such proteases are shown in Table 1 below:

TABLE 1
Specific proteases and their properties

| Protease | Specificity of the cleavage |
|---|---|
| Pepsin | Phe [symbol] X, Leu [symbol] X and pairs of nonpolar amino acids |
| Trypsin | Arg [symbol] X, Lys [symbol] X |
| Chymotrypsin | Tyr [symbol] X, Phe [symbol] X, Trp [symbol] X |
| Thrombin | Arg [symbol] X |
| Thermolysin | X [symbol] Leu, X [symbol] Phe, other non-polar residues |
| Endoproteinase Lys-C | Lys [symbol] X |
| Endoproteinase Glu-C | Glu [symbol] X (partly also Asp [symbol] X) |
| Endoproteinase Arg-C | Arg [symbol] X |
| (clostripain) | |

In contrast to specific proteases, non-specific proteases cleave the peptide chain before or after a whole series of amino acids and thus generate much smaller cleavage pieces. Some examples of such non-specific proteases are shown in Table 2 below:

TABLE 2
Non-specific proteases and their properties

| Protease | Specificity of the cleavage |
|---|---|
| Alkaline protease | Phe [symbol] X, Leu [symbol] X, Val [symbol] X, Ile [symbol] X, Trp [symbol] X, Tyr [symbol] X |
| Papaine | Arg [symbol] X, Lys [symbol] X, Phe [symbol] X |
| Proteinase K | Broad substrate range |

Certain chemicals can be used for the proteolytic analysis of peptides in addition to proteases. Cyanbromide (BrCN) is the most important reagent of this type and cleaves proteins on the C-terminal side of methionine residues. Peptidyl-homoserine lactone is formed in the process. The resulting peptide fragments can then be separated in polyacrylamide gel and visualised by means of staining.

TABLE 3
Typical reagents for chemical proteolysis and their properties

| Reagent | Specificity of the cleavage |
|---|---|
| Cyanbromide | Met [symbol] X |
| Partial acid hydrolysis | Asp [symbol] Pro |
| Hydroxylamine | Asn [symbol] Gly |

A special case of proteolysis is what is known as limited proteolysis. In contrast to total hydrolysis, protein digestion is not complete in this method, but takes place under precisely defined reaction conditions (proteolysis thus proceeds in a limited manner). In the globular regions of a native protein, the peptide bonds are much less exposed than the peptide bonds on the surface of the protein. This means that if a protease is applied for a short time, or if the protease concentration is very low, the individual protein domains may be separated first before the protease reaches cleavage sites further inside. This means that limited proteolysis is carried out with protease dilution and/or suboptimal reaction conditions and is stopped after a short time. In this method, the protein is cleaved proteolytically in its native form (and not after denaturation); this sometimes has consequences for the choice of protease. Metalloprotease cannot be used for digestion if the protein requires e.g. EDTA for optimum stability.

Further information on protein analysis and procedures for proteolytic cleavage of polypeptides can be found in the textbook “Arbeitsmethoden der Biochemie” (‘Working methods in biochemistry’) by Alfred Pingoud, Claus Urbanke, Walter de Gruyter, for example chapter 5.1.6.

In a further embodiment of the invention, the polypeptide has at least 80%, in particular at least 90%, in particular at least 95% sequence identity with one of the domains 1 to 14 of the LEKTI, SPINK6 or SPINK9 polypeptide, especially from the group consisting of SEQ ID Nos 1 to 35, 37, 39 and 40 and 42. Preferably, the polypeptide has at least 80%, in particular at least 90%, in particular at least 95% sequence identity with one of the domains 2 to 14 of the LEKTI, SPINK6 or SPINK9 polypeptide, especially from the group consisting of SEQ ID Nos 2 to 35, 37, 39 and 40 and 42. Particularly, the polypeptide has at least 80%, in particular at least 90%, in particular at least 95% sequence identity with one of the domains 3 to 14 of the LEKTI, SPINK6 or SPINK9 polypeptide, especially from the group consisting of SEQ ID Nos 3 to 35, 37, 39 and 40 and 42.

In still another embodiment, the polypeptide is present in substantially aqueous solution, in particular in aqueous solution with pharmaceutical excipients.

The excipients may be added individually or in combination not only to the polypeptide but also to a final formulation. The excipients may be added at various points in the galenical preparation of the medicinal product.

It may be advisable to stabilise the polypeptide against aggregation or oligomerisation. If necessary, a bacteriostatic agent such as benzyl alcohol, a surfactant such as Tween 20, an isotonic agent such as mannitol, one or more stabilising amino acids such as lysine or arginine, and an antioxidant may also be added to the formulation.

In another embodiment, the polypeptide is formulated for parenteral administration.

In another embodiment, the polypeptide is present in an amount of 1 micromolar to 50 micromolar, preferably 2 micromolar to 25 micromolar, especially preferred 6 micromolar to 12 micromolar per dosage unit.

In the following, the present invention will be further explained in way of examples in a non-limiting manner.

### EXAMPLES

1. Testing of LEKTI Domains for Anti SARS-CoV-2 Activity Using VSV (Vesicular Stomatitis Virus) Based Pseudoparticles

A replication-deficient virus carrying a foreign viral glycoprotein was used as a viral pseudotype.

10,000 Caco2 (colorectal carcinoma cells) were seeded in 100 μl of respective medium. The next day, the medium was removed and replaced by 60 μl of serum-free Caco2 medium. 20 μl of purified LEKTI preparations were added to the cells and incubated for 2 h at 37° C.

Cells were infected with 20 μl of VSV-based pseudoparticles (VSV(Luc-eGFP)) carrying a SARS-CoV-2 spike (VSV(Luc-eGFP)-CoV-2) or VSV-G glycoprotein ((VSV(Luc-eGFP)-G). Infection was measured after 16 h.

EK1 or CM (camostat mesilate) preparations were added as controls instead of LEKTI. These are antiviral agents. EK1 is a peptide inhibitor of virus/cell fusion, while camostat mesilate is a synthetic proteinase inhibitor that inhibits, for example, the proteinase TMPRSS2 (the “corona spike protein activator”).

The infection rate was evaluated in dependency from the concentration of the LEKTI domain, and EK 1 and CM respectively. After, the half maximal inhibitory concentration (IC₅₀) was calculated. LD6 and LD6(III) were toxic at the (two) maximum concentrations tested. The toxic concentrations of LD6 and LD6(III) were not considered when determining the IC50 value. The results are shown in table 4 below:

TABLE 4

| | IC50 | IC50 | |
|---|---|---|---|
| | VSV(Luc-eGFP)-CoV-2 | VSV(Luc-eGFP)-G | unit |
| LD2/3 | 0.4113 | 0.2573 | μM |
| LD6 | 4.091 | 1.609 | |
| LD6(III) | 2.084 | 1.220 | |
| LD15 | 1.848 | 1.021 | |
| EK1 | 0.2811 | >20 | |
| CM | 0.08749 | 433925 | |

LD: recombinant LEKTI domain
EK 1: peptide inhibitor
CM: Camostat mesylate

LD2/3 was a combination of LEKTI domains 2 and 3 (SEQ ID No. 2 and 3), in which the amino acids of LD3 directly follow the amino acids of LD2. LD6 and LD 6(III) correspond to SEQ ID No 17 and 18 respectively. LD15 corresponds to SEQ ID No. 36.

2. Testing of LEKTI Domains for Anti SARS-CoV-2 Activity Using Lentivirus Based Pseudoparticles (LV Based Pseudoparticles)

Replication-deficient viruses carrying a foreign viral glycoprotein were used as viral pseudotypes.

10,000 Caco2 (colorectal carcinoma cells) were seeded in 100 μl of the respective medium. The following day, the medium was removed and replaced with 60 μl of serum-free Caco2 medium.

20 μl of purified LEKTI preparations were added to the cells and incubated for 1 h at 37° C. Cells were infected with 20 μl of LV-based pseudoparticles carrying a SARS-CoV-2 spike (LV(Luc)-CoV-2) or VSV-G glycoprotein (LV(Luc)-G) or MLV glycoprotein (LV(Luc)-MLV). Infection was measured after 48 h.

The infection rate was evaluated in dependency from the concentration of the LEKTI domain, and EK 1 and CM respectively. After, the half maximal inhibitory concentration (IC₅₀) was calculated. LD2/3, LD6, LD6(III) and LD15 were toxic at the (two) maximum concentrations tested. The toxic concentrations, which were determined visually, were not considered when determining IC50. The results are shown in table 5 below:

TABLE 5

| | IC50 | IC50 | IC50 | |
|---|---|---|---|---|
| | LV(Luc)-CoV-2 | LV(Luc)-G | LV(Luc)-MLV | |
| LD2/3 | 0.1808 | 0.1275 | 0.3428 | μM |
| LD6 | 1.034 | 2.146 | unstable | |
| LD6(III) | 1.177 | 0.5006 | 1.710 | |
| LD15 | 0.9556 | 0.5453 | 1.274 | |
| EK1 | 0.08241 | | >20 | |
| CM | 0.2424 | unstable | unstable | |

1. Polypeptide having an amino acid sequence selected from at least 70% sequence identity to any one of domains 1 to 14 of LEKTI, SPINK6 or SPINK9-polypeptide for use in the treatment of a viral infection with a virus, in the infection of which serine proteinases are involved, and slowing down the spread of Infection of said virus.

2. Polypeptide according to claim 1 having an amino acid sequence selected from at least one amino acid sequences SEQ ID Nos 1 to 35, 37, 39, and 40 and 42.

3. Polypeptide according to claim 1, wherein the polypeptide is a homo- or heterodimer from the domains of the LEKTI polypeptide or SPINK6 or SPINK9.

4. Polypeptide according to claim 1, wherein the polypeptide comprises LEKTI, SPINK6 or SPINK9 sequences of porcine, bovine, or LEKTI, SPINK6 or SPINK9 forms of primates, in particular humans.

5. Polypeptide according to claim 1, wherein the polypeptide is a LEKTI, SPINK6 or SPINK9 fragment, a mutant and/or a derivative of the human LEKTI, SPINK6 or SPINK9 domains and has a therapeutic effect.

6. Polypeptide according to claim 1, wherein the polypeptide has at least 80% sequence Identity with one of the domains 1 to 14 of the LEKTI, SPINK6 or SPINK9 polypeptide, in particular from the group consisting of SEQ ID Nos 1 to 20, 21, 23 and 24 and 26.

7. Polypeptide according to claim 1, wherein the virus is an influenza virus or a coronavirus.

8. Polypeptide according to claim which is present in substantially aqueous solution, in particular in aqueous solution with pharmaceutical excipients.

9. Polypeptide according to claim 1, wherein the polypeptide is formulated for parenteral administration.

10. Polypeptide according to claim 1, wherein the polypeptide is present in an amount of from 6 molar to 12 micromolar per dosage unit.

11. A method of using a polypeptide having an amino acid sequence selected from at least 70% sequence Identity to any one of domains 1 to 14 of LEKTI, SPINK6 or SPINK9-polypeptide for manufacturing a medicament in the treatment of a viral Infection with a virus, in the Infection of which serine proteinases are involved, and slowing down the spread of Infection of said virus.

12. The method according to claim 11, wherein the virus is an influenza virus or a coronavirus.

13. The method according to claim 11, wherein the polypeptide has at least 80%, sequence Identity with one of the domains 1 to 14 of the LEKTI, SPINK6 or SPINK9 polypeptide, in particular from the group consisting of SEQ ID Nos 1 to 20, 21, 23 and 24 and 26.

14. A method for treatment of a disease caused by a viral infection with a virus, in the infection of which serine proteinases are involved, and slowing down the spread of Infection of said virus, by administering a polypeptide having an amino acid sequence selected from at least 70% sequence Identity to any one of domains 1 to 14 of LEKTI, SPINK6 or SPINK9-polypeptide.

15. The method according to claim 14, wherein the virus is an influenza virus or a coronavirus, in particular CoV-2.

16. The method according to claim 14, wherein the polypeptide has at least 80% sequence Identity with one of the domains 1 to 14 of the LEKTI, SPINK6 or SPINK9 polypeptide, in particular from the group consisting of SEQ ID Nos 1 to 20, 21, 23 and 24 and 26.

17. Polypeptide according to claim 1, wherein the polypeptide has at least 90% sequence Identity with one of the domains 1 to 14 of the LEKTI, SPINK6 or SPINK9 polypeptide, in particular from the group consisting of SEQ ID Nos 1 to 20, 21, 23 and 24 and 26.

18. Polypeptide according to claim 7, wherein the virus is CoV-2.

19. The method according to claim 11, wherein the polypeptide has at least 90% sequence Identity with one of the domains 1 to 14 of the LEKTI, SPINK6 or SPINK9 polypeptide, in particular from the group consisting of SEQ ID Nos 1 to 20, 21, 23 and 24 and 26.

20. The method according to claim 12, wherein the virus is CoV-2.


SEQUENCE LISTING (42 sequences)

SEQ ID NO: 1 - protein, 57 aa, human
KNEDQEMCHEFQAFMKNGKLFCPQDKKFFQSLDGIMFINKCATCKMILEKEAKSQKR

SEQ ID NO: 2 - protein, 76 aa, human
ARHLARAPKATAPTELNCDDFKKGERDGDFICPDYYEAVCGTDGKTYDNRCALCAENAKTGSQIGVKSEGECKSSN

SEQ ID NO: 3 - protein, 64 aa, human
PEQDVCSAFRPFVRDGRLGCTRENDPVLGPDGKTHGNKCAMCAELFLKEAENAKREGETRIRRN

SEQ ID NO: 4 - protein, 64 aa, human
PEQDVCSAFRPFVRDGRLGCTRENDPVCGPDGKTHGNKCAMCAELFLKEAENAKREGETCIRRN

SEQ ID NO: 5 - protein, 72 aa, human
AEKDFCKEYEKQVRNGRLFCTRESDPVRGPDGRMHGNKCALCAEIFKQRFSEENSKTDQNLGKAEEKTKVKR

SEQ ID NO: 6 - protein, 72 aa, human
AEKDFCKEYEKQVRNGRLFCTRESDPVCGPDGRMHGNKCALCAEIFKQRFSEENSKTDQCLGKAEEKTKVKR

SEQ ID NO: 7 - protein, 72 aa, human
AEKDFCKEYEKQVRNGRLFCTRESDPVRGPDGRMHGNKCALCAEIFKRRFSEENSKTDQNLGKAEEKTKVKR

SEQ ID NO: 8 - protein, 72 aa, human
AEKDFCKEYEKQVRNGRLFCTRESDPVCGPDGRMHGNKCALCAEIFKRRFSEENSKTDQCLGKAEEKTKVKR

SEQ ID NO: 9 - protein, 64 aa, human
EIVKLCSQYQNQAKNGILFCTRENDPIRGPDGKMHGNLCSMCQAYFQAENEEKKKAEARARNKR

SEQ ID NO: 10 - protein, 64 aa, human
EIVKLCSQYQNQAKNGILFCTRENDPICGPDGKMHGNLCSMCQAYFQAENEEKKKAEARCRNKR

SEQ ID NO: 11 - protein, 70 aa, human
ESGKATSYAELCSEYRKLVRNGKLACTRENDPIQGPDGKVHGNTCSMCEVFFQAEEEEKKKKEGKSRNKR

SEQ ID NO: 12 - protein, 70 aa, human
ESGKATSYAELCSEYRKLVRNGKLACTRENDPICGPDGKVHGNTCSMCEVFFQAEEEEKKKKEGKCRNKR

SEQ ID NO: 13 - protein, 70 aa, human
ESGKATSYAELCNEYRKLVRNGKLACTRENDPIQGPDGKVHGNTCSMCEVFFQAEEEEKKKKEGKSRNKR

SEQ ID NO: 14 - protein, 70 aa, human
ESGKATSYAELCNEYRKLVRNGKLACTRENDPICGPDGKVHGNTCSMCEVFFQAEEEEKKKKEGKCRNKR

SEQ ID NO: 15 - protein, 70 aa, human
ESGKATSYAELCSEYRKLVRNGKLACTRENDPIQGPDGKVHGNTCSMCEVFFQAEEEEKKKKEGESRNKR

SEQ ID NO: 16 - protein, 70 aa, human
ESGKATSYAELCSEYRKLVRNGKLACTRENDPICGPDGKVHGNTCSMCEVFFQAEEEEKKKKEGECRNKR

SEQ ID NO: 17 - protein, 70 aa, human
ESGKATSYAELCNEYRKLVRNGKLACTRENDPIQGPDGKVHGNTCSMCEVFFQAEEEEKKKKEGESRNKR

SEQ ID NO: 18 - protein, 70 aa, human
ESGKATSYAELCNEYRKLVRNGKLACTRENDPICGPDGKVHGNTCSMCEVFFQAEEEEKKKKEGECRNKR

SEQ ID NO: 19 - protein, 64 aa, human
QSKSTASFEELCSEYRKSRKNGRLFCTRENDPIQGPDGKMHGNTCSMCEAFFQQEERARAKAKR

SEQ ID NO: 20 - protein, 71 aa, human
EAAKEICSEFRDQVRNGTLICTREHNPVRGPDGKMHGNKCAMCASVFKLEEEEKKNDKEEKGKVEAEKVKR

SEQ ID NO: 21 - protein, 71 aa, human
EAAKEICSEFRDQVRNGTLICTREHNPVCGPDGKMHGNKCAMCASVFKLEEEEKKNDKEECGKVEAEKVKR

SEQ ID NO: 22 - protein, 65 aa, human
EAVQELCSEYRHYVRNGRLPCTRENDPIEGLDGKIHGNTCSMCEAFFQQEAKEKERAEPRAKVKR

SEQ ID NO: 23 - protein, 65 aa, human
EAVQELCSEYRHYVRNGRLPCTRENDPICGLDGKIHGNTCSMCEAFFQQEAKEKERAEPRCKVKR

SEQ ID NO: 24 - protein, 64 aa, human
EAEKETCDEFRRLLQNGKLFCTRENDPVRGPDGKTHGNKCAMCKAVFQKENEERKRKEEEDQRN

SEQ ID NO: 25 - protein, 64 aa, human
EAEKETCDEFRRLLQNGKLFCTRENDPVCGPDGKTHGNKCAMCKAVFQKENEERKRKEEECQRN

SEQ ID NO: 26 - protein, 74 aa, human
AAGHGSSGGGGGNTQDECAEYREQMKNGRLSCTRESDPVRDADGKSYNNQCTMCKAKLEREAERKNEYSRSRSN

SEQ ID NO: 27 - protein, 74 aa, human
AAGHGSSGGGGGNTQDECAEYREQMKNGRLSCTRESDPVCDADGKSYNNQCTMCKAKLEREAERKNEYSRSCSN

SEQ ID NO: 28 - protein, 74 aa, human
AAGHGSSGGGGGNTQDECAEYQEQMKNGRLSCTRESDPVRDADGKSYNNQCTMCKAKLEREAERKNEYSRSRSN

SEQ ID NO: 29 - protein, 74 aa, human
AAGHGSSGGGGGNTQDECAEYQEQMKNGRLSCTRESDPVCDADGKSYNNQCTMCKAKLEREAERKNEYSRSCSN

SEQ ID NO: 30 - protein, 68 aa, human
GTGSESGKDTCDEFRSQMKNGKLICTRESDPVRGPDGKTHGNKCTMCKEKLEREAAEKKKKEDEDRSN

SEQ ID NO: 31 - protein, 68 aa, human
GTGSESGKDTCDEFRSQMKNGKLICTRESDPVCGPDGKTHGNKCTMCKEKLEREAAEKKKKEDECRSN

SEQ ID NO: 32 - protein, 79 aa, human
TGERSNTGERSNDKEDLCREFRSMQRNGKLICTRENNPVRGPYGKMHINKCAMCQSIFDREANERKKKDEEKSSSKPSN

SEQ ID NO: 33 - protein, 79 aa, human
TGERSNTGERSNDKEDLCREFRSMQRNGKLICTRENNPVCGPYGKMHINKCAMCQSIFDREANERKKKDEECSSSKPSN

SEQ ID NO: 34 - protein, 67 aa, human
NAKDECSEFRNYIRNNELICPRENDPVHGADGKFYTNKCYMCRAVFLTEALERAKLQEKPSHVRASQ

SEQ ID NO: 35 - protein, 67 aa, human
NAKDECSEFRNYIRNNELICPRENDPVCGADGKFYTNKCYMCRAVFLTEALERAKLQEKCSHVRASQ

SEQ ID NO: 36 - protein, 87 aa, human
EEDSPDSFSSLDSEMCKDYRVLPRIGYLCPKDLKPVCGDDGQTYNNPCMLCHENLIRQTNTHIRSTGKCEESSTPGTTAASMPPSDE

SEQ ID NO: 37 - protein, 80 aa, human
MKLSGMFLLLSLALFCFLTGVFSQGGQVDCGEFQDPKVYCTRESNPHCGSDGQTYGNKCAFCKAIVKSGGKISLKHPGKC

SEQ ID NO: 38 - protein, 23 aa, human
MKLSGMFLLLSLALFCFLTGVFS

SEQ ID NO: 39 - protein, 57 aa, human
QGGQVDCGEFQDPKVYCTRESNPHCGSDGQTYGNKCAFCKAIVKSGGKISLKHPGKC

SEQ ID NO: 40 - protein, 86 aa, human
MRATAIVLLLALTLATMFSIECAKQTKQMVDCSHYKKLPPGQQRFCHHMYDPICGSDGKTYKNDCFFCSKVKKTDGTLKFVHFGKC

SEQ ID NO: 41 - protein, 19 aa, human
MRATAIVLLLALTLATMFS

SEQ ID NO: 42 - protein, 67 aa, human
IECAKQTKQMVDCSHYKKLPPGQQRFCHHMYDPICGSDGKTYKNDCFFCSKVKKTDGTLKFVHFGKC